(19) United States
(12) Patent Application Publication
(10) Pub. No.: US 2021/0058611 A1
(43) Pub. Date: Feb. 25, 2021

(54) MULTIVIEWING VIRTUAL REALITY USER INTERFACE

(71) Applicant: INTERDIGITAL CE PATENT HOLDINGS, Paris (FR)

(72) Inventor: William G Redmann, Glendale, CA (US)

(21) Appl. No.: 16/964,643

(22) PCT Filed: Jan. 31, 2019
(86) PCT No.: PCT/IB2019/000135

§ 371 Date: Jul. 24, 2020

Related U.S. Application Data
(60) Provisional application No. 62/625,620, filed on Feb. 2, 2018.

(51) Int. Cl. H04N 13/368; H04N 13/344; G06F 3/0481

(52) U.S. Cl. CPC H04N 13/368; G06T 19/006; G06F 3/04815; H04N 13/344

(57) ABSTRACT

A system, user interface and method is provided for receiving input regarding movement of a user as registered by a sensor and providing images, at least partially based on the input to an interior display and an exterior display of a head mounted display (HMD) or the like. The interior display is disposed so as to be viewable only to the user wearing the HMD, and the exterior display is viewable by at least one other observer that is not the user. The external display can facilitate social interaction, enhance training, and provide for monitoring the virtual activities of the user.

### TECHNICAL FIELD

The present disclosure relates generally to user interfaces and in particular to Virtual Reality (VR) or Augmented Reality (AR) user interfaces allowing with multi-viewing capabilities.

### BACKGROUND

This section is intended to introduce the reader to various aspects of art, which may be related to various aspects of the present disclosure that are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In recent years, immersive experiences created by Virtual Reality (VR) and Augmented Reality (AR) devices have become the subject of increased attention. This is because VR/AR can be used practically in every field to perform various functions including testing, entertaining, training, and teaching. For example, engineers and architects can use VR/AR in modelling of new designs. Doctors can use VR/AR technologies to practice and perfect difficult operations ahead of time and military experts can develop strategies by simulating battlefield operations. VR/AR is also used extensively in the gaming and entertainment industries to provide interactive experiences and enhance audience enjoyment. VR/AR enables the creation of a simulated environment that feels real and can accurately duplicate experiences in real or imaginary worlds.

While VR/AR offers unique experiences, most of the usages provide for single and solitary experiences. This drawback gives such experiences an antisocial aspect, and could stigmatize the technology. In addition, in cases that require an observer to assist the user of the VR/AR system such as during training exercises, the inability to share experiences provide challenges. Consequently, multiplayer and multi-shared environments are desirable that can embrace a more social VR/AR world.

### SUMMARY

A system, user interface, and method is provided for sending input regarding movement of a housing worn by a user as registered by a sensor to a controller. An interior first display and an exterior second display are provided and disposed such that said first display is only viewable by a user. The second display is viewable by at least one other observer that is not the user. In some embodiments the second display is not viewable by the user. In some embodiments a sensor is provided for registering a movement corresponding to the first display. At least one controller is configured for receiving input from the sensor, and providing images to the first display and the second display at least partially based on the input, wherein the input is representative of the movement of at least one of the user, the first display and the second display.

Additional features and advantages are realized through similar techniques and other embodiments and aspects are described in detail herein and are considered a part of the claimed embodiments. For a better understanding of the embodiments with advantages and features, refer to the description and to the drawings.

### BRIEF DESCRIPTION OF THE DRAWINGS

The present disclosure will be better understood and illustrated by means of the following embodiment and execution examples, in no way limitative, with reference to the appended figures on which:

FIG. 1 schematically represents a functional overview of an encoding and decoding system according one or more embodiments of the disclosure;

FIG. 2 schematically represents a system, according to one embodiment;

FIG. 3 schematically represents a system, according to another embodiment;

FIG. 4 schematically represents a system, according to another embodiment;

FIG. 5 schematically represents a system, according to another embodiment;

FIG. 6 schematically represents a system, according to another embodiment

FIG. 7 schematically represents a system, according to another embodiment;

FIG. 8 schematically represents a system according to another embodiment,

FIG. 9 schematically represents a system according to another embodiment,

FIG. 10 schematically represents an immersive video rendering device according to an embodiment,

FIG. 11 schematically represents an immersive video rendering device according to another embodiment,

FIG. 12 schematically represents an immersive video rendering device according to another embodiment,

FIG. 13 schematically represents a user interface having a first interior and a second exterior display according one embodiment;

FIG. 14 provides a more detailed view of embodiment of FIG. 13, according to one embodiment;

FIG. 15 provides an alternate embodiment to that of FIG. 14;

FIG. 16 provides an alternative embodiment having a video projector, according to another embodiment;

FIG. 17 provides an alternative embodiment having smart mobile devices, according to another embodiment;

FIG. 18 is an illustration of a VR/AR wearable device view according to one embodiment;

FIG. 19 is a flow chart representation of a methodology for providing users and observers multiple perspective views according to one embodiment; and

Wherever possible, the same reference numerals will be used throughout the figures to refer to the same or like parts.

### DESCRIPTION

It is to be understood that the figures and descriptions of the present embodiments have been simplified to illustrate elements that are relevant for a clear understanding of the present embodiments, while eliminating, for purposes of clarity, many other elements found in typical digital multimedia content delivery methods and systems. However, because such elements are well known in the art, a detailed discussion of such elements is not provided herein. The disclosure herein is directed to all such variations and modifications known to those skilled in the art.

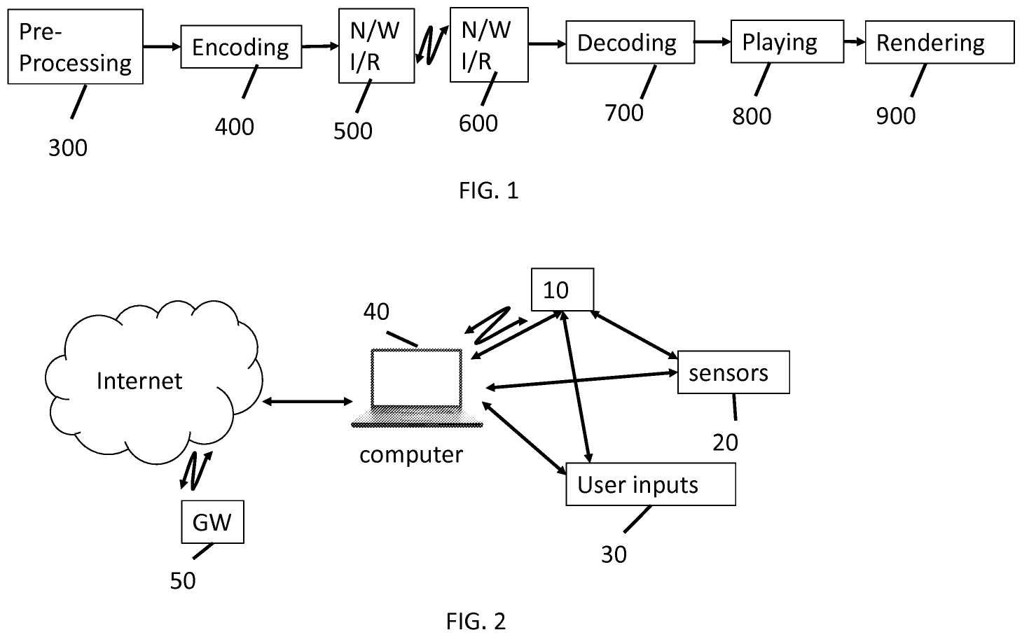

FIG. 1 schematically illustrates a general overview of an encoding and decoding system according to one or more embodiments. The system of FIG. 1 is configured to perform one or more functions. A pre-processing module 300 may be provided to prepare the content for encoding by an encoding device 400. The pre-processing module 300 may perform multi-image acquisition, merging of the acquired multiple images in a common space (for example, a 3D sphere from which the direction to each pixel is encoded, by a mapping into a 2D frame using, for example, but not limited to, an equirectangular mapping or a cube mapping). The pre-processing module 300 might instead acquire an omnidirectional video in a particular format (for example, equi-rectangular) as input, and pre-process the video to change the mapping into a format more suitable for encoding. Depending on the acquired video data representation, the pre-processing module 300 may perform a mapping space change. Another implementation might combine the multiple images into a common space having a point cloud representation. Encoding device 400 packages the content in a form suitable for transmission and/or storage for recovery by a compatible decoding device 700. In general, though not strictly required, the encoding device 400 provides a degree of compression, allowing the common space to be represented more efficiently (i.e., using less memory for storage and/or less bandwidth required for transmission). In the case of a 3D sphere mapped onto a 2D frame, the 2D frame is effectively an image that can be encoded by any of a number of image (or video) codecs. In the case of a common space having a point cloud representation, the encoding device 400 may provide point cloud compression, which is well known, e.g., by octree decomposition. After being encoded, the data, which may be encoded immersive video data or 3D CGI encoded data for instance, are sent to a network interface 500, which may be typically implemented in any network interface, for instance present in a gateway. The data are then transmitted through a communication network 550, such as internet but any other network may be foreseen. Then the data are received via network interface 600. Network interface 600 may be implemented in a gateway, in a television, in a set-top box, in a head mounted display (HMD) device, in an immersive (projective) wall or in any immersive video rendering device. After reception, the data are sent to a decoding device 700. Decoded data are then processed by a player 800. Player 800 prepares the data for the rendering device 900 and may receive external data from sensors or users input data. More precisely, the player 800 prepares the part of the video content that is going to be displayed by the rendering device 900. The decoding device 700 and the player 800 may be integrated in a single device (e.g., a smartphone, a game console, a STB, a tablet, a computer, etc.). In another embodiment, the player 800 may be integrated in the rendering device 900.

Various types of systems may be used to perform functions of an immersive display device, for rendering an immersive video or an interactive immersive experience (e.g., a VR game). Embodiments of a system, for processing augmented reality (AR) or virtual reality (VR) content are illustrated in FIGS. 2 to 9. Such systems are provided with one or more processing functions, and include an immersive video rendering device which may comprise a head-mounted display (HMD), a tablet, or a smartphone for example, and may optionally include one or more sensors. The immersive video rendering device may also include interface modules between the display device and one or more modules performing the processing functions. The presentation processing functions may be integrated into the immersive video rendering device or performed by one or more processing devices. Such a processing device may include one or more processors and a communication interface with the immersive video rendering device, such as a wireless or wired communication interface.

The processing device may also include a communication interface (e.g., 600) with a wide access network such as internet and access content located on a cloud, directly or through a network device such as a home or a local gateway. The processing device may also access a local storage device (not shown) through an interface such as a local access network interface (not shown), for example an Ethernet type interface. In an embodiment, the processing device may be provided in a computer system having one or more processing units. In another embodiment, the processing device may be provided in a smartphone which can be connected by a wired link or a wireless link to the video to change the mapping into a format more suitable for encoding. Depending on the acquired video data representation, the pre-processing module 300 may perform a mapping space change. After being encoded, the data, which may be encoded immersive video data, or 3D CGI encoded data for instance, are sent to a network interface 500, which may be typically implemented in any network interface, for instance present in a gateway. The data are then transmitted through a communication network, such as internet but any other network may be foreseen. Then the data are received via network interface 600. Network interface 600 may be implemented in a gateway, in a television, in a set-top box, in a head mounted display device, in an immersive (projective) wall or in any immersive video rendering device. After reception, the data are sent to a decoding device 700. Decoded data are then processed by a player 800. Player 800 prepares the data for the rendering device 900 and may receive external data from sensors or users input data. More precisely, the player 800 prepares the part of the video content that is going to be displayed by the rendering device 900. The decoding device 700 and the player 800 may be integrated in a single device (e.g., a smartphone, a game console, a STB, a tablet, a computer, etc.). In another embodiment, the player 800 may be integrated in the rendering device 900.

An “immersive content” often refers to a video or other streamed content or images, commonly encoded as a rectangular frame that is a two-dimension array of pixels (i.e., element of color information) like a “regular” video or other form of image content. In many implementations, the following processes may be performed for presentation of that immersive content. To be rendered, the two-dimensional frame is, first, mapped on the inner face of a convex volume, also referred to as mapping surface (e.g. a sphere, a cube, a pyramid), and, second, a part of this volume is captured by a virtual camera. Images captured by the virtual camera are displayed by the screen of the immersive display device. In some embodiments, stereoscopic video is provided and decoding results in one or two rectangular frames, which can be projected onto two mapping surfaces, one for each of the user's eyes, a portion of which are captured by two virtual cameras according to the characteristics of the display device.

Pixels in the content appear to the virtual camera(s) according to a mapping function from the frame. The mapping function depends on the geometry of the mapping surface. For a same mapping surface (e.g., a cube), various mapping functions are possible. For example, the faces of a cube may be structured according to different layouts within the frame surface. A sphere may be mapped according to an equirectangular projection or to a gnomonic projection for example. The organization of pixels resulting from the selected projection function may modify or break line continuities, orthonormal local frame, pixel densities and may introduce periodicity in time and space. These are typical features that are used to encode and decode videos. Typically, today, there is a lack of taking specificities of immersive videos into account in encoding and decoding methods. Indeed, as immersive videos are 360° videos, a panning, for example, introduces motion and discontinuities that require a large amount of data to be encoded while the content of the scene does not change. Taking immersive videos specificities into account while encoding and decoding video frames would bring valuable advantages to the state-of-art methods.

In another embodiment, the system includes an auxiliary device which communicates with the immersive video rendering device and with the processing device. In such an embodiment, the auxiliary device may perform at least one of the processing functions. The immersive video rendering device may include one or more displays. The device may employ optics such as lenses in front of each display. The display may also be a part of the immersive display device such as for example in the case of smartphones or tablets. In another embodiment, displays and optics may be embedded in a helmet, in glasses, or in a wearable visor. The immersive video rendering device may also include one or more sensors, as described later, for use in the rendering. The immersive video rendering device may also include interfaces or connectors. It may include one or more wireless modules in order to communicate with sensors, processing functions, handheld or devices or sensors related to other body parts.

When the processing functions are performed by the immersive video rendering device, the immersive video rendering device can be provided with an interface to a network directly or through a gateway to receive and/or transmit content.

The immersive video rendering device may also include processing functions executed by one or more processors and configured to decode content or to process content. By processing content here, it is understood functions for preparing content for display. This may include, for instance, decoding content, merging content before displaying it and modifying the content according to the display device.

One function of an immersive content rendering device is to control a virtual camera which captures at least a part of the content structured as a virtual volume. The system may include one or more pose tracking sensors which totally or partially track the user's pose, for example, the pose of the user's head, in order to process the pose of the virtual camera. One or more positioning sensors may be provided to track the displacement of the user. The system may also include other sensors related to the environment for example to measure lighting, temperature or sound conditions. Such sensors may also be related to the body of a user, for instance, to detect or measure sweating or heart rate. Information acquired through these sensors may be used to process the content. The system may also include user input devices (e.g. a mouse, a keyboard, a remote control, a joystick). Information from user input devices may be used to process the content, manage user interfaces or to control the pose of the virtual camera (or an actual camera). Sensors and user input devices communicate with the processing device and/or with the immersive rendering device through wired or wireless communication interfaces.

An embodiment of the immersive video rendering device 10, will be described in more detail with reference to FIG. 10. The immersive video rendering device includes a display 101. The display is, for example an OLED or LCD type display. The immersive video rendering device 10 is, for instance a HMD, a tablet, or a smartphone. The device 10 may include a touch sensitive surface 102 (e.g. a touchpad or a tactile screen), a camera 103, a memory 105 in connection with at least one processor 104 and at least one communication interface 106. The at least one processor 104 processes the signals received from the sensor(s) 20 (FIG. 2). Some of the measurements from sensors are used to compute the pose of the device and to control the virtual camera. Sensors which may be used for pose estimation include, for instance, gyroscopes, accelerometers or compasses. In more complex systems, a rig of cameras for example may also be used. The at least one processor 104 performs image processing to estimate the pose of the device 10. Some other measurements may be used to process the content according to environmental conditions or user reactions. Sensors used for detecting environment and user conditions include, for instance, one or more microphones, light sensor or contact sensors. More complex systems may also be used such as, for example, a video camera tracking eyes of a user. In such a case the at least one processor performs image processing to perform the expected measurement. Data from sensor(s) 20 and user input device(s) 30 may also be transmitted to the computer 40 which will process the data according to the input of the sensors.

Memory 105 includes parameters and code program instructions for the processor 104. Memory 105 may also include parameters received from the sensor(s) 20 and user input device(s) 30. Communication interface 106 enables the immersive video rendering device to communicate with the computer 40 FIG. 2). The Communication interface 106 of the processing device may include a wireline interface (for example a bus interface, a wide area network interface, a local area network interface) or a wireless interface (such as a IEEE 802.11 interface or a Bluetooth® interface). Computer 40 sends data and optionally control commands to the immersive video rendering device 10. The computer 40 processes the data, for example to prepare the data for display by the immersive video rendering device 10. Processing may be carried out exclusively by the computer 40 or part of the processing may be carried out by the computer and part by the immersive video rendering device 10. The computer 40 is connected to internet, either directly or through a gateway or network interface 50. The computer 40 receives data representative of an immersive video from the internet, processes these data (for example. decode the data and may prepare the part of the video content that is going to be displayed by the immersive video rendering device 10) and sends the processed data to the immersive video rendering device 10 for display. In another embodiment, the system may also include local storage (not represented) where the data representative of an immersive video are stored, said local storage may be on the computer 40 or on a local server accessible through a local area network for instance (not represented).

Embodiments of a first type of system for displaying augmented reality, virtual reality, augmented reality (also mixed reality) or any content from augmented reality to virtual reality will be described with reference to FIGS. 2 to 6. In one embodiment, these are combined with a large field-of-view content that can provide up to a 360 degree view of a real, fictional or mixed environment. This large field-of-view content may be, among others, a three-dimension computer graphic imagery scene (3D CGI scene), a point cloud, streaming content or an immersive video or panoramic picture or images. Many terms may be used to define technology that provides such content or videos such as for example Virtual Reality (VR), Augmented Reality (AR) 360, panoramic, 4π, steradians, omnidirectional, immersive and alongside large-field-of-view as previously indicated.

FIG. 2 schematically illustrates an embodiment of a system configured to decode, process and render immersive videos. The system includes an immersive video rendering device 10, one or more sensors 20, one or more user input devices 30, a computer 40 and a gateway 50 (optional).

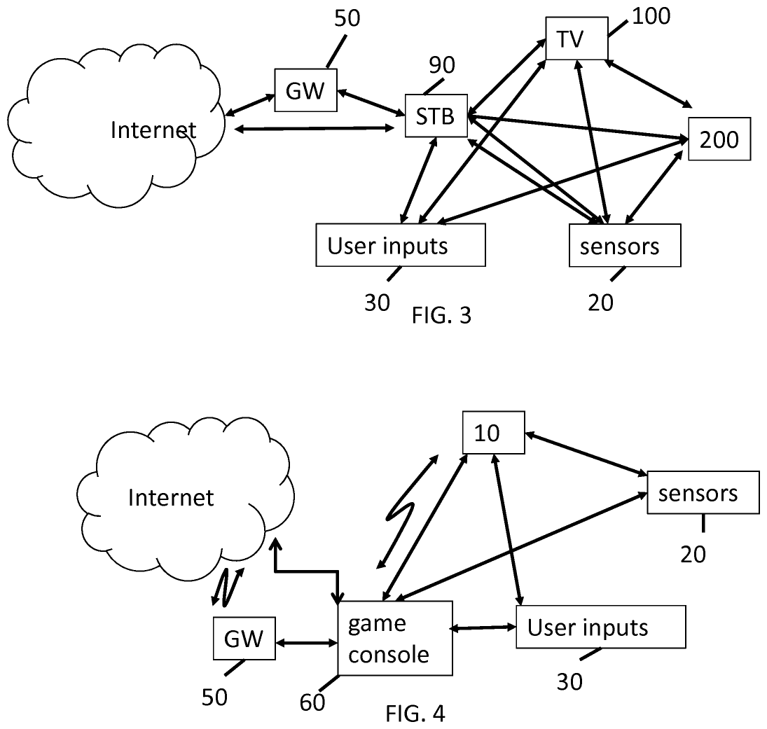

FIG. 3 schematically represents a second embodiment of a system configured to decode, process and render immersive videos. In this embodiment, an STB 90 is connected to a network such as internet directly (i.e. the STB 90 includes a network interface) or via a gateway 50. The STB 90 is connected through a wireless interface or through a wired interface to a rendering device such as a television set 100 or an immersive video rendering device 200. In addition to classic functions of an STB, STB 90 includes processing functions to process video content for rendering on the television 100 or on any immersive video rendering device 200. These processing functions are similar to the processing functions described for computer 40 and are not described again here. Sensor(s) 20 and user input device(s) 30 are also of the same type as the sensor(s) and input device(s) described earlier with reference to FIG. 2. The STB 90 obtains the data representative of the immersive video from the internet. In another embodiment, the STB 90 obtains the data representative of the immersive video from a local storage (not represented) where the data representative of the immersive video are stored.

FIG. 4 schematically represents a third embodiment of a system configured to decode, process and render immersive videos. In the third embodiment a game console 60 processes the content data. Game console 60 sends data and optionally control commands to the immersive video rendering device 10. The game console 60 is configured to process data representative of an immersive video and to send the processed data to the immersive video rendering device 10 for display. Processing may be done exclusively by the game console 60 or part of the processing may be done by the immersive video rendering device 10.

The game console 60 is connected to internet, either directly or through a gateway or network interface 50. The game console 60 obtains the data representative of the immersive video from the internet. In another embodiment, the game console 60 obtains the rendering device 10. Processing may be carried out exclusively by the computer 40 or part of the processing may be carried out by the computer and part by the immersive video rendering device 10. The computer 40 is connected to internet, either directly or through a gateway or network interface 50. The computer 40 receives data representative of an immersive video from the internet, processes these data (for example. decode the data and may prepare the part of the video content that is going to be displayed by the immersive video rendering device 10) and sends the processed data to the immersive video rendering device 10 for display. In another embodiment, the system may also include local storage (not represented) where the data representative of an immersive video are stored, said local storage may be on the computer 40 or on a local server accessible through a local area network for instance (not represented).

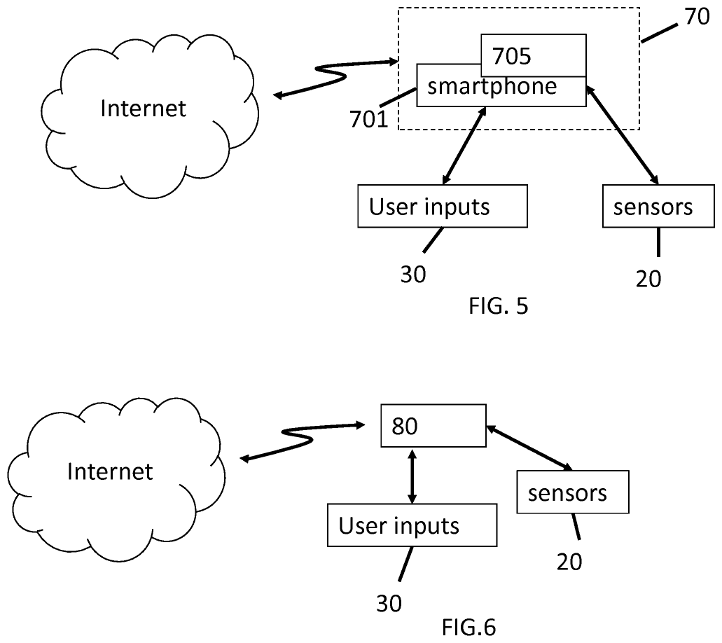

FIG. 5 schematically represents a fourth embodiment of a system configured to decode, process and render immersive videos the immersive video rendering device 70 is provided by a smartphone 701 inserted in a housing 705. The smartphone 701 may be connected to internet and thus may obtain data representative of an immersive video from the internet. In another embodiment, the smartphone 701 obtains data representative of an immersive video from a local storage (not represented) where the data representative of an immersive video are stored, said local storage may be on the smartphone 701 or on a local server accessible through a local area network for instance (not represented).

FIG. 6 schematically represents a fifth embodiment of the first type of system in which the immersive video rendering device 80 includes functionalities for processing and displaying the data content. The system includes an immersive video rendering device 80, sensors 20 and user input devices 30. The immersive video rendering device 80 is configured to process (e.g. decode and prepare for display) data representative of an immersive video possibly according to data received from the sensors 20 and from the user input devices 30. The immersive video rendering device 80 may be connected to Internet and thus may obtain data representative of an immersive video from the internet. In another embodiment, the immersive video rendering device 80 obtains data representative of an immersive video from a local storage (not represented) where the data representative of an immersive video are stored, said local storage may be provided on the rendering device 80 or on a local server accessible through a local area network for instance (not represented).

An embodiment of immersive video rendering device 80 is illustrated in FIG. 12. The immersive video rendering device includes a display 801, for example an OLED or LCD type display, a touchpad (optional) 802, a camera (optional) 803, a memory 805 in connection with at least one processor 804 and at least one communication interface 806. Memory 805 includes parameters and code program instructions for the processor 804. Memory 805 may also include parameters received from the sensors 20 and user input devices 30. Memory 805 may have a large enough capacity to store data representative of the immersive video content. Different types of memories may provide such a storage function and include one or more storage devices such as a SD card, a hard disk, a volatile or non-volatile memory . . . ) Communication interface 806 enables the immersive video rendering device to communicate with internet network. The processor 804 processes data representative of the video to display images on display 801. The camera 803 captures images of the environment for an image processing step. Data are extracted from this step to control the immersive video rendering device.

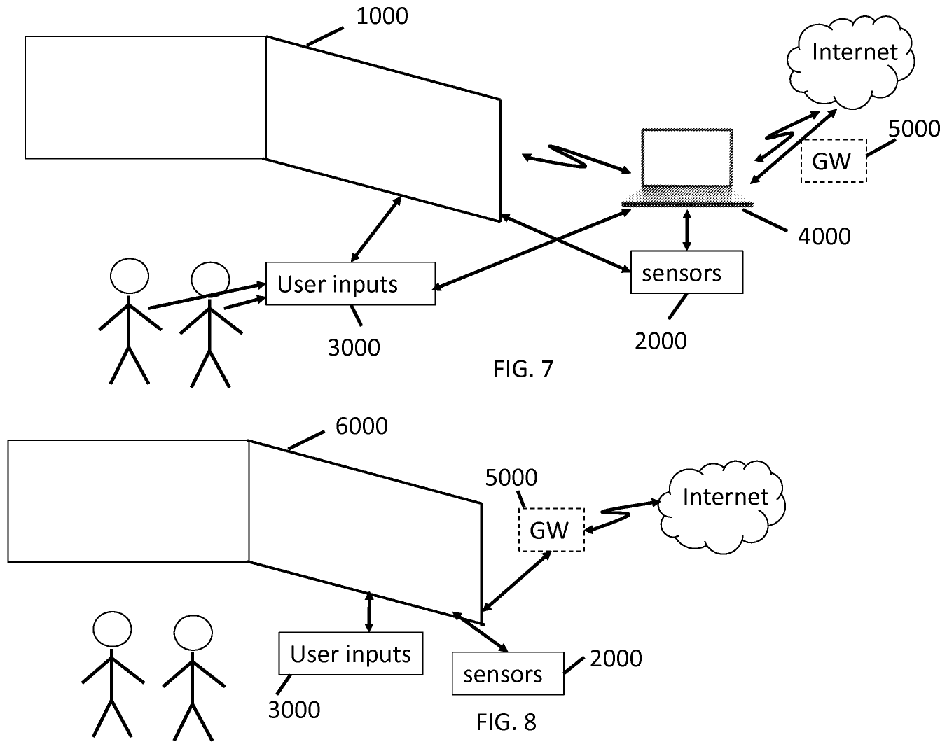

Embodiments of a second type of system, for processing augmented reality, virtual reality, or augmented virtuality content are illustrated in FIGS. 7 to 9. In these embodiments the system includes an immersive wall or CAVE (a recursive acronym for “CAVE Automatic Virtual Environment”).

FIG. 7 schematically represents an embodiment of the second type of system including a display 1000—an immersive (projective) wall which receives data from a computer 4000. The computer 4000 may receive immersive video data from the internet. The computer 4000 can be connected to internet, either directly or through a gateway 5000 or network interface. In another embodiment, the immersive video data are obtained by the computer 4000 from a local storage (not represented) where data representative of an immersive video are stored, said local storage may be in the computer 4000 or in a local server accessible through a local area network for instance (not represented).

This system may also include one or more sensors 2000 and one or more user input devices 3000. The immersive wall 1000 may be an OLED or LCD type, or a projection display, and may be equipped with one or more cameras (not shown). The immersive wall 1000 may process data received from the more or more sensors 2000. The data received from the sensor(s) 2000 may, for example, be related to lighting conditions, temperature, environment of the user, e.g., the position of objects, and the position of a user. In some cases, the imagery presented by immersive wall 1000 may be dependent upon the position of a user, for example to adjust the parallax in the presentation.

The immersive wall 1000 may also process data received from the one or more user input devices 3000. The user input device(s) 3000 may send data such as haptic signals in order to give feedback on the user emotions. Examples of user input devices 3000 include for example handheld devices such as smartphones, remote controls, and devices with gyroscope functions.

Data may also be transmitted from sensor(s) 2000 and user input device(s) 3000 data to the computer 4000. The computer 4000 may process the video data (e.g. decoding them and preparing them for display) according to the data received from these sensors/user input devices. The sensors signals may be received through a communication interface of the immersive wall. This communication interface may be of Bluetooth type, of WIFI type or any other type of connection, preferentially wireless but may also be a wired connection.

Computer 4000 sends the processed data and, optionally, control commands to the immersive wall 1000. The computer 4000 is configured to process the data, for example prepare the data for display by the immersive wall 1000. Processing may be done exclusively by the computer 4000 or part of the processing may be done by the computer 4000 and part by the immersive wall 1000.

FIG. 8 schematically represents another embodiment of the second type of system. The system includes an immersive (projective) wall 6000 which is configured to process (for example decode and prepare data for display) and display the video content and further includes one or more sensors 2000, and one or more user input devices 3000.

The immersive wall 6000 receives immersive video data from the internet through a gateway 5000 or directly from internet. In another embodiment, the immersive video data are obtained by the immersive wall 6000 from a local storage (not represented) where the data representative of an immersive video are stored, said local storage may be in the immersive wall 6000 or in a local server accessible through a local area network for instance (not represented).

This system may also include one or more sensors 2000 and one or more user input devices 3000.The immersive wall 6000 may be of OLED or LCD type and be equipped with one or more cameras. The immersive wall 6000 may process data received from the sensor(s) 2000 (or the plurality of sensors 2000). The data received from the sensor(s) 2000 may for example be related to lighting conditions, temperature, environment of the user, such as position of objects.

The immersive wall 6000 may also process data received from the user input device(s) 3000. The user input device(s) 3000 send data such as haptic signals in order to give feedback on the user emotions. Examples of user input devices 3000 include for example handheld devices such as smartphones, remote controls, and devices with gyroscope functions.

The immersive wall 6000 may process the video data (e.g. decoding them and preparing them for display) according to the data received from these sensor(s)/user input device(s). The sensor signals may be received through a communication interface of the immersive wall. This communication interface may include a Bluetooth® type, a WIFI type or any other type of wireless connection, or any type of wired connection. The immersive wall 6000 may include at least one communication interface to communicate with the sensor(s) and with the internet.

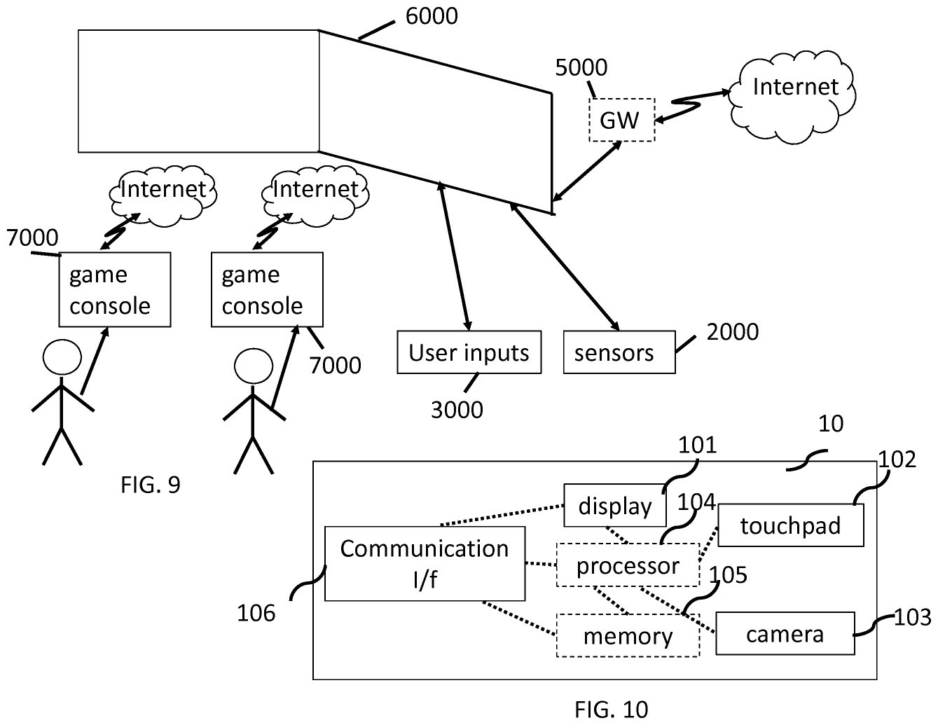

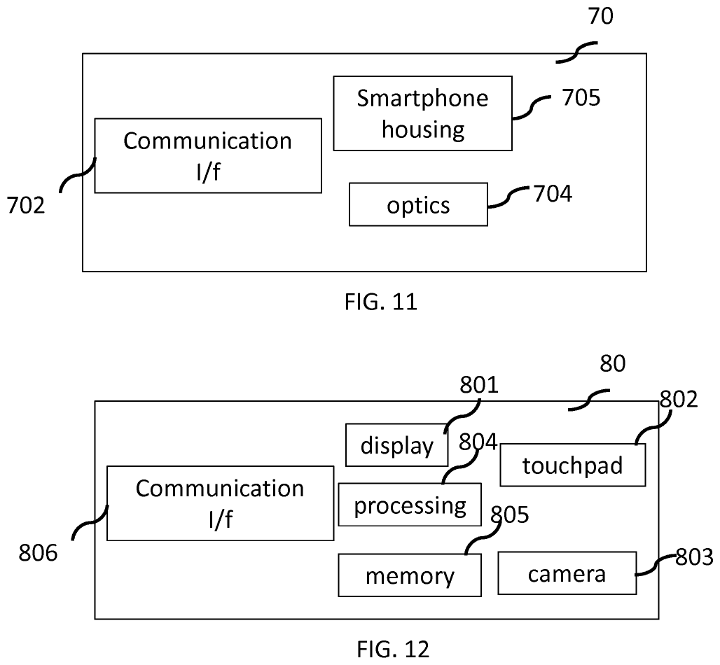

FIG. 9 illustrates another embodiment in which an immersive wall is used for gaming. One or more gaming consoles 7000 are connected, for example through a wireless interface to the immersive wall 6000. The immersive wall 6000 receives immersive video data from the internet through a gateway 5000 or directly from internet. In an alternative embodiment, the immersive video data are obtained by the immersive wall 6000 from a local storage (not represented) where the data representative of an immersive video are stored, said local storage may be in the immersive wall 6000 or in a local server accessible through a local area network for instance (not represented).

Gaming console 7000 sends instructions and user input parameters to the immersive wall 6000. Immersive wall 6000 processes the immersive video content, for example, according to input data received from sensor(s) 2000 and user input device(s) 3000 and gaming console(s) 7000 in order to prepare the content for display. The immersive wall 6000 may also include internal memory to store the content to be displayed.

In a VR or AR environment, there is content surrounding the user wearing a head-mounted display. However, at the same time, it is very easy for the user to miss interesting or exciting events if the user is looking in the wrong direction. This problem also exists when the user is viewing 360° video content on a TV or screen-based computing device. It is desired to provide a user a physical remote to pan the view space by varying degrees so that content corresponding to different angles can be provided. Since most prior art cannot provide such content, an issue arises in a number of applications. In addition, even when content can be provided accordingly, it will be desirable to bring attention of the user to crucial information that the user may miss due to inattention.

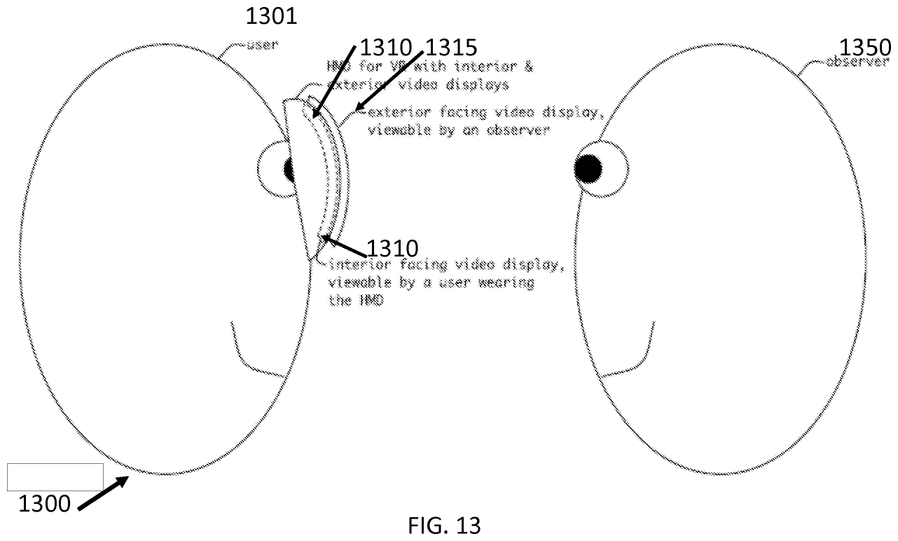

FIGS. 13 to 19 provides different embodiments displaying VR/AR user interfaces and systems and methodology. Conventionally, in many VR/AR systems, a display system is worn on the head of a user. In some cases, the display is driven by a controller that is presenting pre-recorded video (e.g., 360° video) with real time manipulation to render a view corresponding to user movement and the field(s) of view subtended by the display. In other cases, the display is driven by the controller rendering computer-generated video in real time. In both cases, the video presented is based, at least in part, on the movement of the user, most commonly changes in the orientation of the user's head. In such systems, the user is often the only one to see the video images produced, in which case an observer sees the movements of the user, but doesn't receive any information about what the user is seeing, and thus doesn't know what the user is reacting to. This offers a problem for an observer who is assisting the user, for example in a training exercise, e.g., a user learning to use the VR equipment or equipment or learning a skill where the VR system is merely a learning tool. This is also a disadvantage to friends or family members watching another playing with the VR system—it's hard to share the experience if only one party is seeing the video.

In one example, a scenario can be imagined when parents and children are the users of the AR/VR systems. Many parents cannot appreciate what the child is viewing or experiencing when using most VR/AR user interfaces, including head mounted displays (hereinafter HMDs). In such an example, a children's program may have ended and segued into a scary program that is not age appropriate. In one embodiment as provided in FIGS. 13 to 18, the parents can both appreciate and even monitor what a child is watching. In some configurations, a copy of the video in the HMD can be sent to a remote display, in which case an observer can watch the remote display, but in such circumstances, they typically look away from the user wearing the VR system and what is being shown on the remote display is no longer has the context of the user's posture or movements.

It should be noted that while HMDs are used as way of example in the present description, all VR/AR user interfaces can be used with the present embodiments and HMDs are used only to ease understanding as can be appreciated by those skilled in the art.

In many conventional AR/VR systems the user wears an HMD and there is a remote display for use by other than the user. The remote display is typically static and the user is typically moving, it becomes difficult to correlate what the user is doing with what is on the screen, due to the divided spatial relationship. Therefore, in one embodiment, by attaching an external display to the HMD, the presentation of video to the user can be mirrored to an outside surface of the HDM for intuitive observation of the user's experience. In some embodiments, the image on the outside can differ from the image on the inside, e.g., to better map to field of view included in the image to the shape and size of the exterior display, or to enlarge the central region of the interior image to better indicate on the exterior display what the user is paying attention to. The addition of an exterior mounted display on an HMD allows an observer to see what is going on in the virtual world that the user is experiencing, with an intuitive presentation corresponding to what the user is seeing. The video on the external facing display can further be annotated or augmented with information that may not be available to the user wearing the HMD, for example, indications of heart rate or estimates of cumulative stress on the user, or hints about points of interest not immediately in view (which can induce the observer to communicate these hints to the user wearing the HMD, thereby extending the interactive experience to the observer and making the experience more social).

The image shown on the interior display may be mirrored when shown on the exterior display. This can be either the same image shown as a mirror image, or a separate image that is a mirror image of the original. To be clear, by “mirror image” is meant an image that is flipped left-to-right. In a mirror image, any text in the original image would reads backwards in the mirror image. Text appearing in the image shown on the interior display would be reversed by such mirroring on the exterior display, so in some embodiments that separately create images for the interior and exterior displays, the rendering of text is reversed in an otherwise mirrored image so that the text appears right-reading for the observer.

FIG. 13 provides such an example. The system 1300 provides for a case where a user 1301 utilizing a VR/AR user interface 1302 (i.e. here a wearable HMD) is being watched by an observer 1350 not wearing the HMD nor having a similar user interface. In this particular example, the user interface or the HMD has an interior display 1310 viewable by the user 1301 when the user is wearing the HMD. The HMD also has an exterior display viewable by the observer. The interior display 1310 of the HMD operates in the manner well-known. For clarity, optics, e.g., lenses, necessary for user 1301 to view the interior display 1310, are not shown. The exterior display 1315 is supplied with a video signal corresponding to what is shown on the interior display 1310.

The video signal to the exterior display 1315 may be identically the video signal provided to the interior display 1310, where the video signal is shown in reverse (flipped left-to-right) by the exterior display. In other embodiments, the video signal to the exterior display may be a distinct, but still corresponding, video signal. In some embodiments, this distinct video signal represents an image that is a mirror image of the image represented by the video signal provided to the interior display 1310. In still other embodiments, the distinct video signal represents an image that is a mirror image of that for the interior display, but in which text has been reversed to be right-reading in the mirror image as described above.

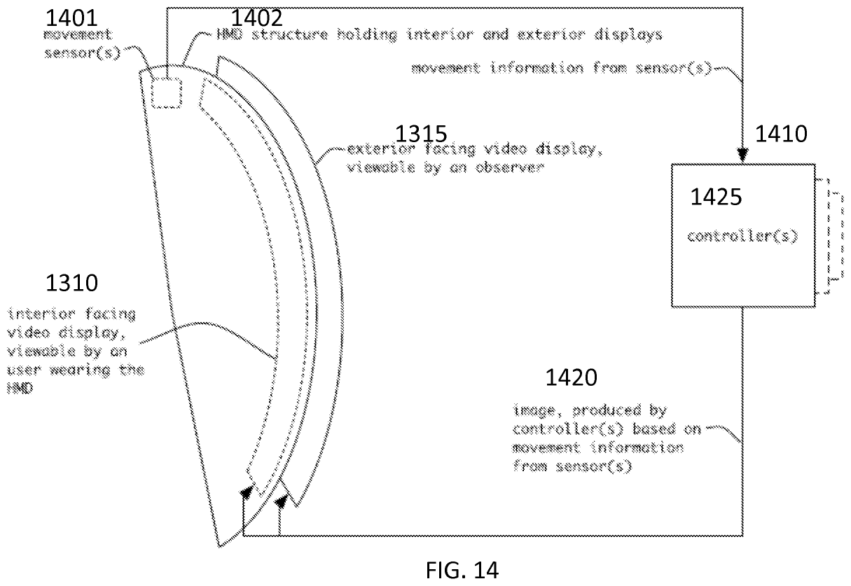

FIG. 14 shows a block diagram of the embodiment shown in FIG. 13 where the displays are part of an HMD. In this example, the HMD structure supports an interior facing display 1310 and an exterior facing display 1315. The interior facing display is viewable by a user when the HMD is worn, while the exterior facing display is viewable by an observer not wearing the HMD as was previously illustrated in FIG. 13. In this embodiment, one or more movement sensors 1401 supply movement information 1410, typically at least orientation information, to one or more controllers 1425. The one or more controllers 1425 produce an image, represented by image signal 1420, based on the movement information. The image signal is provided both the interior and exterior displays, with the exterior display presenting the image reversed left-to-right with respect the presentation of the interior display so that there is a correspondence of handedness between both presentation. By way of example, if the image provided by the controller contained an arrow pointing to the left, presentation of that image to the user wearing the HMD might cause the user to turn toward the left. Were the same image presented on the exterior display without being flipped horizontally, that arrow would point to the user's right, and the user would appear to be turning in a direction opposite to the one indicated. If the exterior display flips the image horizontally (i.e., presents a mirrored image), then the arrow as shown on the exterior display and the arrow as shown on the interior display are pointing in similar directions, making the exterior presentation correspond to and be consistent with the interior presentation.

In one embodiment, special optics is not necessary for proper viewing of the interior facing display and not shown in the figures but if desirable such can be provided. However, many HMDs provide a Fresnel lens or other optical system to allow the user's eyes to focus on the interior facing display. For HMDs that employ light field, or holographic, or other display technologies to present an image to a user wearing the HMD, these are contemplated by this description and included in the designation herein of the interior display. Also, while no mechanisms are illustrated in the figures to secure the user interface to a user (i.e., HMD to a wearing user's head), as can be appreciated by those skilled in the art, a variety of configurations can be made to provide a secure arrangement, including but not limited to use of head bands, caps, ear hooks (as is typical for eyeglasses), counterweights, and the like, as these are quite diverse and not affected by the present embodiments. Additionally, the present embodiments includes in the notion of “being worn” that a user might just be holding the HMD to their face without addition straps or such to maintain that position (e.g., as with Google Cardboard).

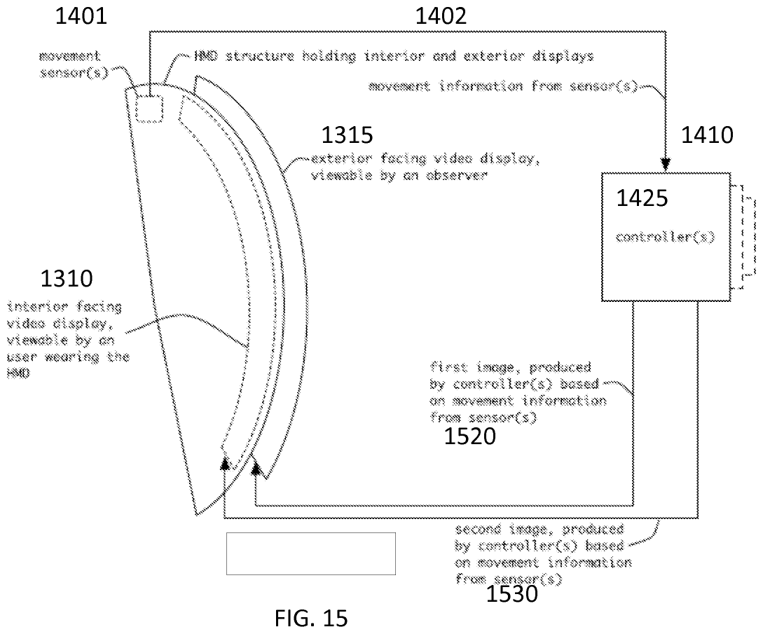

FIG. 15 shows an alternate where the controller(s) 1425 provide two distinct images represented by image signals 1520 and 1530, one for the interior display 1310 (signal 1530) and one for the exterior display 1315 (signal 1520). In the simplest version of this embodiment, the controllers merely provide a first image to the interior facing display and a second image that is a mirrored image of the first image to the exterior facing display. In alternate versions of this embodiment, the second image can be different (e.g., represent a wider or narrower field of view than the first image, and/or the second image can be differently annotated, etc.)

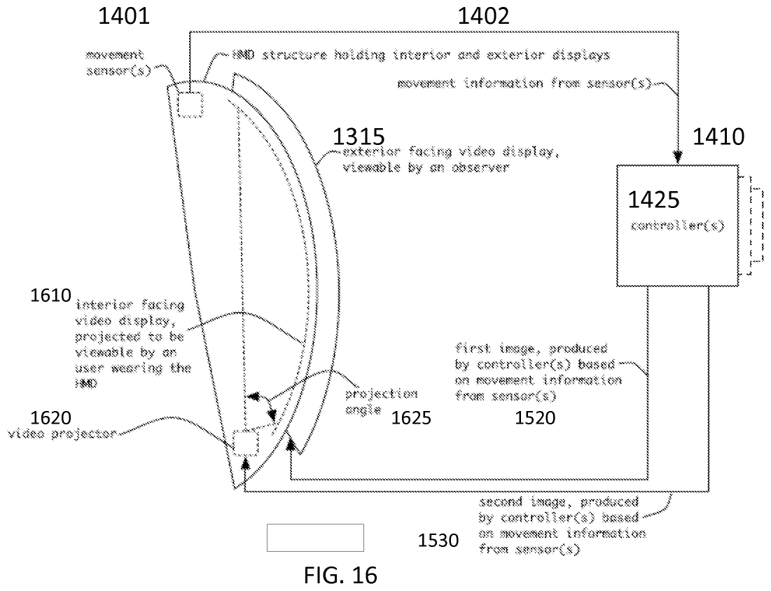

FIG. 16 is similar to FIG. 15, but in this alternate embodiment, the interior facing display 1610 is embodied as a projector 1620 onto a screen that forms the viewable portion of the display. Again, note that any conventional viewing optics that may be necessary for a user to focus on the interior display while wearing the HMD are not shown. The projection angle 1625 can be selectively arranged for best viewing results.

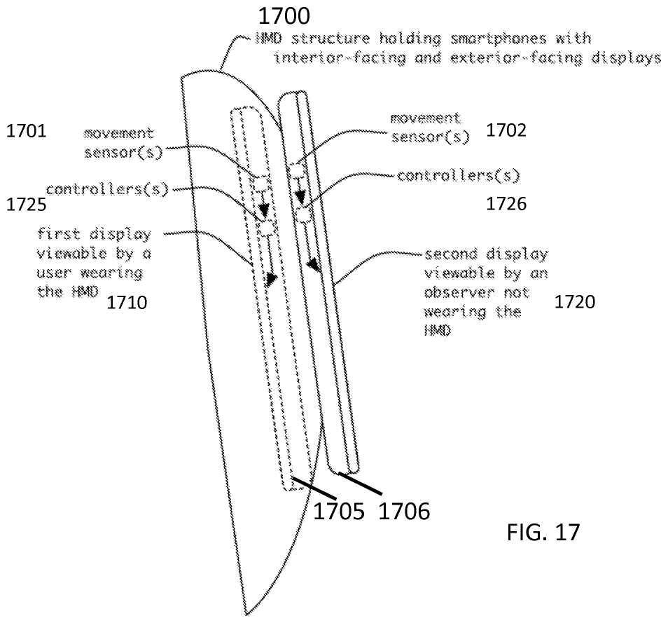

In yet another embodiment, as shown in FIG. 17, an HDM structure 1700 supports two separate presentation devices: One inward facing (1705) and one outward facing (1706), wherein the support for each presentation device is either direct or indirect (i.e., where one presentation device was to mount to the structure 1700 and the second presentation device mounts to either the structure directly, or indirectly by mounting to the first presentation device). Here the two presentation devices are shown to be implemented as smartphones (although many other arrangements can be also possible as appreciated by those skilled in the art), each containing both a movement sensor and controller (1701,1702 and 1725 and 1726 respectively), to drive their respective displays 1710 and 1720, much as described above. For example, in the embodiment discussed, a first display 1710 of a first smartphone faces a user wearing the HMD 1700 and is seen by the user through viewing optics (not shown). A second display 1720 of a second smartphone faces away from the user, making it viewable by an observer watching the user. The second smartphone reveals a representation of the user's experience as observers are watching the user, allowing the observer to better understand, share the event, and to some degree share in the experience. The two presentation devices can share a communication link (not shown) to aid in synchronization. This link can be via an radio frequently link, e.g., Near Field Communication (NFC), wireless local area network (WLAN or WiFi), or personal area network (PAN or Bluetooth™), This link can be via an audio synchronization, e.g., an app on one of the smartphones makes a beep or other sound (which may be ultrasonic) through a speaker (not shown) and an app on the other smartphone detects that beep through a microphone (not shown), thereby marking a common point in time with an accuracy of less than 1 mS (based on the time sound takes to travel between the spacing from the beeping phones' speaker to the detecting phone's microphone, and the variable internal latencies of each smartphone's audio processing (e.g., buffering, packetizing). Alternatively, the user could press a start button (not shown) on each of the two smartphones simultaneously.

In some embodiments, the exterior display and the structure for head mounting the exterior display may be distinct and independent of the structure used to head mount the first display. For example, a first HMD for VR use without an external screen capability can be independently provided to be worn by a user. Separately, an external screen is provided with the appropriate structure or attachments to mount the second screen to the user's head either directly or indirectly (i.e., the second screen forms a second HMD, but one that does not face the user when worn; or, the second screen with or without additional structure attaches to the first HMD, e.g., by clipping or clamping onto the first HMD, or being adhered to it). Given that the external facing display represents a mass that is added onto the HMD and might reduce comfort over long periods of use, the external facing display may be removable for those situations where an observer is not present, or is present, but doesn't need to see the user experience reflected. Further, removing the external display, or merely disabling it, can provide the advantage of reduced power consumption.

As described herein, the image provided to the external display is generally representative of what the user is seeing on the internal display. In an alternative embodiment, the image provided to the external display can be different, or be exaggerated. For example, if the user is hiding from a pirate and is behind a rock in the virtual world presented by the HMD, the user might be presented with a view of the rock on the interior display while one the external display the observer might be able to see that the pirate through the rock (as if a reflection of the user's vantage if the user had x-ray vision) or the observer might be able to see the back of the pirate, the external view calculated as if the situation were being view at some distance (e.g., 20 feet) from the user in the virtual world, but looking back toward the user in the virtual world, generally back along the line of the user's direction of gaze. Such a view for the observer can enable different kinds of interactions (e.g., He's coming around the rock! Move behind the tree on your left!”), thereby increasing the social nature of the experience.

A HMD is said to be “operably positioned” when the HMD is worn or held to the face of a user, such that the user, through the appropriate viewing optics of the HMD, is able to see and focus on the interior display of the HMD. When “operably positioned” an HMD shares the frame of reference of the user's head, that is, in contact with or otherwise holds a position relative to the user's head, where if the user's head turns, the HMD likewise turns, such that the display remains fixed with respect to the viewer's skull, give or take a bit of fixedness if the wearing or holding of the HMD is a little loose. The term “operably positioned” is helpful to describe Google Cardboard VR Viewers and the like, which are generally not strapped to a user's head and worn, but merely held is place, as are old fashioned stereopticons or the classic ViewMaster™ toy.

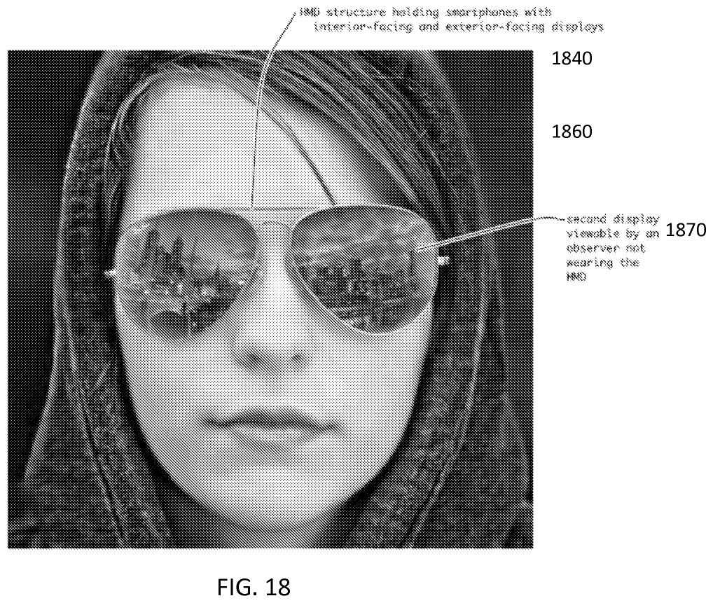

FIG. 18 provides an example as per one embodiment. In FIG. 18, a user 1860 is shown wearing an HDM-style user interface in form of glasses 1840. In this example, the observer (not shown) can look to the user's face and in the external display(s) 1870 see images. In this example, the observer's view is depicted, seeing the user looking out on an evening view of a cityscape, near a river.

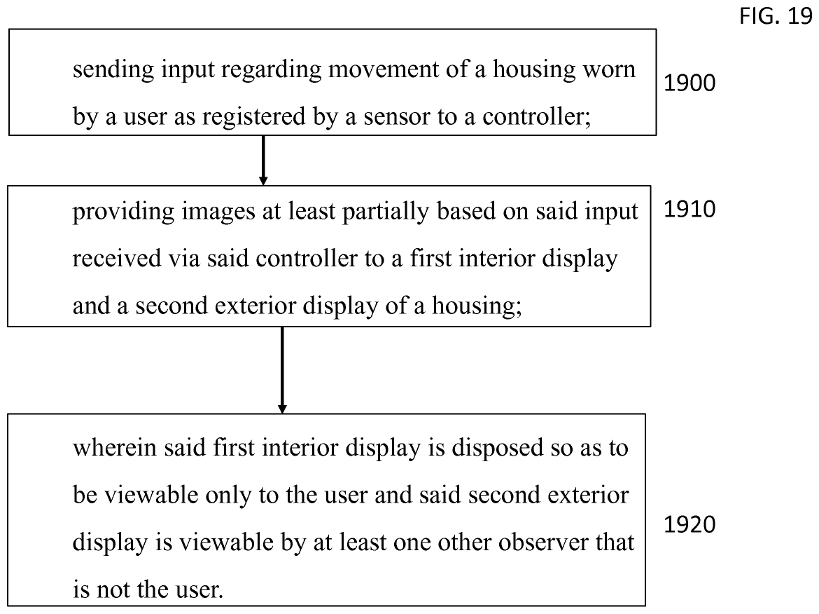

FIG. 19 is a flowchart depiction according to one embodiment. In on embodiment, as shown at step 1900, input is received regarding movement of a housing worn by a user. The input represents the movement of the user as registered via a sensor and is sent to a controller. The movement could be that of the housing itself as opposed to the user per se. In step 1910 at least one image is provided, the at least one image being at least partially based on the input received via the controller. As indicated in step 1920, the interior first display is disposed so as to be viewable only to the user and the exterior second display is viewable by at least one other observer that is not the user. Images are provided to a first interior display and a second exterior display of a housing. The images can be varied, identical or include additional text. The text can also be varied, missing from one display, reversed between the displays, or be identical.

Additionally, audio can be provided to accompany the displayed content, for example through speakers or headphones (not shown) by a signal provided by the controller to be in synchronization with the displayed content. In some embodiments, a common audio program can be audible to both the user and the observer. In an alternative embodiment, the audio provided to the user may be through headphones or other near-field emission not audible, or otherwise not meant for the observer, while audio (which may be the same or different) is separately provided to the user, for example through a speaker audible to the observer, or through another device such as a Bluetooth® earphone or headset having wireless communication with at least one controller. In some cases, the audio presented to the user may be 3D audio (e.g., binaural, or object-based audio that renders individual sounds so as to appear positionally consistent with the visual presentation, even as the user turns). The audio presented to the observer may be independently rendered, and may even be 3D audio too, but if so, then preferably rendered apropos to the facing of the observer as may be estimated from the dominant facing of the user and a predetermined estimate of the observer distance therefrom. In another embodiment, sensors of the HMD may identify an observer's position relative to the user, and this information used to render audio for the observer accordingly.

In some embodiments (not shown), the external display provided for an observer might be atop or behind the user's head, or located elsewhere on the user, e.g., on a backpack, which may be a more convenient position for the observer, depending on the nature of user. For example, in a military or police training exercise, the observer might be a teacher following a student (the user) through a physical encounter environment that the student is exploring. It would be awkward for the observer to backpedal through the environment, particularly when the student might suddenly rush forward or extend a weapon into the space the observer occupies. In such situations, the external display for the observer might be mounted behind the user's head or on the user's back. Note that in this configuration, the image displayed for the user by the interior display could be the same as the image displayed for the observer, as the handedness is consistent for both displays, i.e., an arrow pointing leftward in the video signal would point in substantially the same direction (to the user's left) on both screens.

While some embodiments have been described, it will be understood that those skilled in the art, both now and in the future, may make various improvements and enhancements which fall within the scope of the claims which follow. These claims should be construed to maintain the proper protection for the embodiments first described.

1. A system comprising:
a first display and a second display, disposed such that said first display is viewable at least by a user and said second display is viewable by at least one other observer that is not the user; and,
at least one processor configured for receiving input from a sensor, and providing images to said first display and said second display at least partially based on said input, wherein said input is representative of a movement of at least one of said user, said first display and said second display.

2. A method comprising:
receiving input representative of movement of at least one of a user, ad first display and a second display wherein said first display is disposed so as to be viewable at least to the user and said second display is viewable by at least one observer that is not the user;
providing, images at least partially based on said input to the first display and the second display;

3. The system of claim 1, wherein said first and second displays are provided on a housing, the first display being interior to the housing and the second display being exterior to the housing.

4. The system of claim 1, wherein movement of said first or second display comprise selective movements.

5. The system of claim 1 further comprising a sensor registering said movement.

6. The system of claim 3, wherein said housing is wearable by said user and said movement of corresponds movement of said user.

7. The system of claim 6, wherein said housing includes a pair of glasses, said first interior display includes a display surface facing inwardly toward the user and said second exterior display includes a display surface facing away from the user.

8. The system of claim 6, wherein said housing is a head-mounted display (HMD), said first interior display provides images image toward eye of the user when the HMD is worn and said second exterior display provides images away from the eye of the user.

9. The system of claim 1, wherein said images provided to said first display are identical to images provided to said second exterior display.

10. The system of claim 1, wherein one of said images provided to said first display is different than a corresponding one of said images provided to said second display.

11. The system of claim 9, wherein said images provided to said first display and second display include text or additional information that is not identical and can selectively relate to status of said user.

12. The system of claim 3, wherein said housing includes auditory components for providing sounds to said user and said observer and wherein said sounds provided to said user and said observer are at least partially different.

13. A computer program product comprising instructions which when executed by a processor cause the processor to carry out the method of claim 2.

14. The method of claim 2, wherein said first and second displays are provided on a housing, the first display being interior to the housing and the second display being exterior to the housing.

15. The method of claim 2, wherein movement of said first or second display comprise selective movements.

16. The method of claim 3, wherein said housing is wearable by said user and said movement of corresponds movement of said user.

17. The method of claim 6, wherein said housing includes a pair of glasses, said first interior display includes a display surface facing inwardly toward the user and said second exterior display includes a display surface facing away from the user.

18. The method of claim 6, wherein said housing is a head-mounted display (HMD), said first interior display provides images image toward eye of the user when the HMD is worn and said second exterior display provides images away from the eye of the user.

19. The method of claim 2, wherein said images provided to said first display are identical to images provided to said second exterior display.

20. The method of claim 2, wherein one of said images provided to said first display is different than a corresponding one of said images provided to said second display.

21. The method of claim 9, wherein said images provided to said first display and second display include text or additional information that is not identical and can selectively relate to status of said user.

22. The method of claim 3, wherein said housing includes auditory components for providing sounds to said user and said observer and wherein said sounds provided to said user and said observer are at least partially different.
